(19) United States
(12) Patent Application Publication
(10) Pub. No.: US 2025/0129356 A1
(43) Pub. Date: Apr. 24, 2025

(54) ARGINASE BEARING VESICLE COMPOSITION AND METHOD OF USE

(71) Applicant: CAPRICOR, INC., San Diego, CA (US)

(72) Inventors: Minghao SUN, San Diego, CA (US); Kristi ELLIOTT, Encinitas, CA (US); Li-En HSIEH, La Jolla, CA (US); Mafalda CACCIOTTOLO, San Diego, CA (US); Ciana MORA, San Diego, CA (US); Bailey MURPHY, San Diego, CA (US)

(21) Appl. No.: 18/920,959

(22) Filed: Oct. 20, 2024

Related U.S. Application Data
(60) Provisional application No. 63/695,203, filed on Sep. 16, 2024.
(60) Provisional application No. 63/592,133, filed on Oct. 20, 2023.

(51) Int. Cl. C12N 9/78; A61K 38/00; A61K 47/69; C07K 14/705

(52) U.S. Cl. CPC C12N 9/78; A61K 47/6901; C07K 14/70596; A61K 38/00; C07K 2319/03; C12Y 305/03001

(57) ABSTRACT

Disclosed are compositions containing exosomes loaded with arginase or arginase chimeras for treating hyperammonemia due to arginase deficiency, and methods for treating hyperammonemia by administering compositions containing exosomes loaded with arginase or arginase chimeras.

### CROSS-REFERENCE TO RELATED APPLICATIONS

This application claims priority to U.S. Provisional Application No. 63/592,133, filed Oct. 20, 2023, and U.S. Provisional Application No. 63/695,203, filed Sep. 16, 2024, the contents of which are incorporated herein by reference in their entireties.

### SEQUENCE LISTING

The instant application contains a Sequence Listing which has been submitted electronically in .XML format and is hereby incorporated by reference in its entirety. Said .XML copy, created on Oct. 19, 2024, is named SEQLST_519US.xml and is 14,232 bytes in size. The sequence listing contained in this .XML file is part of the specification and is hereby incorporated by reference herein in its entirety.

### BACKGROUND OF THE INVENTION

Arginase 1 deficiency (A1D) is an autosomal recessive metabolic disorder in which the subject lacks the final enzyme (Arg1), in the urea cycle, the major pathway to detoxify ammonia, leading to hyperammonemia. Arg 1 is expressed mainly in the hepatocytes and catalyzes arginine into ornithine and urea. The clinical manifestations of AID usually appear in late infancy to the second year of life. Symptoms include microcephaly, spasticity, seizures, clonus, loss of ambulation, progressive mental impairment, growth retardation, periodic episodes of hyperammonemia and failure to thrive associated with hyperargininemia.

Current therapies include dietary restrictions such as low-protein arginine-restriction, dialysis to remove excess ammonia, nitrogen scavenger drugs such as sodium phenylacetate and sodium benzoate, and glycerol phenylbutyrate (Ravicti®), a nitrogen-binding adjunctive therapeutic. Despite these therapies and diet and blood management procedures, there exists a significant need for improved effective Arg 1 enzyme replacement therapy to address the underlying etiology.

### SUMMARY OF THE INVENTION

In a first aspect, a pharmaceutical composition for use in treating Arginase 1 deficiency or hyperammonemia containing a vesicle loaded with an arginase activity is provided. FIG. 1.

In one embodiment, the arginase activity is an Arg1 enzyme, ortholog, homolog, or functional fragment thereof. In a specific embodiment, the Arg1 enzyme is a human Arg1 enzyme. In one embodiment, the arginase activity is positioned within the lumen of the vesicle. In one embodiment, the Arg1 enzyme is soluble and not fused to another protein. In one embodiment, the vesicle is an exosome.

In another embodiment, the arginase activity is an Arg1 enzyme, ortholog, homolog, or functional fragment thereof positioned on the outside of the vesicle. In one embodiment, the N-terminus of the arginase activity is fused to a transmembrane domain and a linker which is fused to the C-terminus of a tetraspanin. In a specific embodiment, the Arg1 enzyme is a human Arg1 enzyme. In a specific embodiment, the tetraspanin is a CD9. In a more specific embodiment, the arginase is fused to CD9 and has a primary structure as set forth in SEQ ID NO:1. In one embodiment, the vesicle is an exosome.

In another embodiment, the arginase activity is an Arg1 enzyme, ortholog, homolog, or functional fragment thereof positioned on the outside of the vesicle. In one embodiment, the C-terminus of the arginase activity is fused to a transmembrane domain and a linker which is fused to the N-terminus of a tetraspanin. In a specific embodiment, the Arg1 enzyme is a human Arg1 enzyme. In a specific embodiment, the tetraspanin is a CD9. In a more specific embodiment, the arginase is fused to CD9 and has a primary structure as set forth in SEQ ID NO:2. In one embodiment, the vesicle is an exosome.

In another embodiment, the arginase activity is an Arg1 enzyme, ortholog, homolog, or functional fragment thereof positioned on the inside of the vesicle. In one embodiment, the C-terminus of the arginase activity is fused to a linker which is fused to the N-terminus of a tetraspanin. In a specific embodiment, the Arg1 enzyme is a human Arg1 enzyme. In a specific embodiment, the tetraspanin is a CD9. In a more specific embodiment, the arginase is fused to CD9 and has a primary structure as set forth in SEQ ID NO:3. In one embodiment, the vesicle is an exosome.

In a second aspect, a method for making a pharmaceutical composition for use in treating Arginase 1 deficiency or hyperammonemia containing a vesicle loaded with an arginase activity is provided. In one embodiment, eukaryotic cells are transduced with polynucleotides encoding any one or more of the polypeptide constructs of the first aspect (FIG. 1), the cells express (constitutively or induced) the Arg1 construct(s), media containing vesicles secreted by the transduced cells is collected, and the vesicles are isolated from the conditioned media, purified and concentrated using centrifugation, filtration, and molecular sieving (e.g., size exclusion chromatography). In one embodiment, the Arg1 constructs are constitutively expressed. In another embodiment, the Arg1 constructs are inducibly expressed. In one embodiment, the eukaryotic cells are 293f cells. In one embodiment, the vesicles are exosomes.

In a third aspect, a method of treating Arginase 1 deficiency of hyperammonemia in a subject in need thereof by administering a pharmaceutical composition of any containing a vesicle loaded with an arginase activity is provided. In one embodiment, cells are contacted with one or more pharmaceutical compositions of the first aspect. In one embodiment, one or more pharmaceutical compositions of the first aspect are administered intra venously to a subject in need thereof. In one embodiment, several doses of the one or more pharmaceutical compositions of the first aspect are administered to a subject in need thereof at various times to sustain therapeutic levels of arginase activity in the subject in need thereof.

### BRIEF DESCRIPTION OF THE DRAWINGS

FIG. 1 is a cartoon depicting various Arg1 constructs (numbered 1A, 1B, 1C, and 1D) loaded onto a vesicle. Construct 1A depicts soluble Arg1 depicted as positioned within a vesicle lumen. Construct 1B depicts Arg1 positioned outside the vesicle and fused to the C-terminus of a tetraspanin. Construct 1C depicts Arg1 positioned outside the vesicle and fused to the N-terminus of a tetraspanin. Construct 1D depicts Arg1 positioned inside the vesicle and fused to the N-terminus of a tetraspanin. Throughout the disclosure, construct 1A may be referred to as soluble Arg1 or soluble recombinant Arg1. Throughout the disclosure, construct 1D may be referred to as

FIGS. 2A-2D are western blots of proteins extracted from 293F cells transduced with various Arg1 constructs depicted if FIG. 1 probed for CD9 and Arg1. Here, un-modified 293f cells were transduced to constitutively express Arg1 with different constructs and the expression of Arg1 protein was evaluated by Jess using Arg1 specific antibody. Arg1 expression was confirmed by Jess using both anti-CD9 and anti-Arg1 antibodies.

FIG. 2A is a western blot of cells transduced to express the construct 1A of FIG. 1. Column 1 is molecular weight markers; column 2 is cell extract probed with anti-CD9 antibody; column 3 is cell extract probed with anti-Arg1 antibody; column 4 is recombinant Arg1 probed with anti-Arg1 antibody.

FIG. 2B is a western blot of cells transduced to express the construct 1B of FIG. 1. Column 1 is molecular weight markers; column 2 is cell extract probed with anti-CD9 antibody; column 3 is cell extract probed with anti-Arg1 antibody.

FIG. 2C is a western blot of cells transduced to express the construct 1C of FIG. 1. Column 1 is molecular weight markers; column 2 is cell extract probed with anti-CD9 antibody; column 3 is cell extract probed with anti-Arg1 antibody.

FIG. 2D is a western blot of cells transduced to express the construct 1D of FIG. 1. Column 1 is molecular weight markers; column 2 is cell extract probed with anti-CD9 antibody; column 3 is cell extract probed with anti-Arg1 antibody,

FIG. 3 is a bar graph depicting arginase activity in units per liter (U/L) in cell lines as a function of cell line construct. Here, Arg1 activity was measured in all four Arg1-expressing cell lines using a colorimetric-based Arg1 activity assay and all four cell lines showed high level of Arg1 activity. The top bar represents 1 μg recombinant Arg1 as a positive control; second bar from the top represent negative control; third bar from the top represents 293f cells transformed with Arg1 encoding polynucleotide (the construct 1A depicted in FIG. 1); fourth bar from the top represents 293f cells transformed with Arg1-CD9 fusion encoding polynucleotide (the construct 1B depicted in FIG. 1); fifth bar from the top represents 293f cells transformed with CD9-Arg1-out encoding polynucleotide (the construct 1C depicted in FIG. 1); and sixth bar from the top bar represents 293f cells transformed with CD9-Arg1-in encoding polynucleotide (embodiment depicted in FIG. 1D).

FIGS. 4A and 4B depict FACS analyses line graphs of Arg1 protein in the different Arg1 cell lines harboring the polynucleotide constructs encoding the constructs 1A, 1B, 1C, and 1D of FIG. 1. Surface expression (panel 4A) and internal expression (panel 4B) of Arg1 is presented.

FIG. 4A depicts a FACS plot of surface Arg1 expression as a function of cell line. The top line graph represents an untransformed 293f cell line; the second from top line graph represents a 293f cell line harboring the recombinant Arg1 (construct 1A of FIG. 1); the third from top line graph represents a 293f cell line harboring the recombinant Arg1-CD9-out construct (construct 1B of FIG. 1)); the fourth from top line graph represents a 293f cell line harboring the recombinant CD9-Arg1-out construct (construct 1C of FIG. 1); the fifth from top line graph represents a 293f cell line harboring the recombinant CD9-Arg1-in construct (construct 1D of FIG. 1).

FIG. 4B depicts a FACS plot of intracellular Arg1 expression as a function of cell line. The top line graph represents an untransformed 293f cell line; the second from top line graph represents a 293f cell line harboring the recombinant Arg1 (construct 1A of FIG. 1); the third from top line graph represents a 293f cell line harboring the recombinant Arg1-CD9-out construct (construct 1B of FIG. 1)); the fourth from top line graph represents a 293f cell line harboring the recombinant CD9-Arg1-out construct (construct 1C of FIG. 1); the fifth from top line graph represents a 293f cell line harboring the recombinant CD9-Arg1-in construct (construct 1D of FIG. 1).

FIGS. 5A-5D depict western blots of proteins extracted from 293f cells transduced to express tet-inducible Arg1 with or without doxycycline with different designs (as described in FIG. 1) and probed with an Arg1 specific antibody.

FIG. 5A shows a blot of 293f cells expressing an Arg1-tet construct (construct 1A of FIG. 1). Column 1 is molecular weight markers; column 2 is extracts from cells not treated with doxycycline and probed with anti-Arg1 antibody; column 3 is extracts from cells treated with doxycycline and probed with anti-Arg1 antibody. The band at approximately 42 kDa in columns 2 and 3 represents actin. The band at approximately 40 kDa in column 3 represents Arg 1.

FIG. 5B shows blot of 293f cells expressing a CD9-Arg1-in-tet construct (construct 1D of FIG. 1). Column 1 is molecular weight markers; column 2 is extracts from cells not treated with doxycycline and probed with anti-Arg1 antibody; column 3 is extracts from cells treated with doxycycline and probed with anti-Arg1 antibody. The band at approximately 42 kDa in columns 2 and 3 represents actin. The doublet bands between 40 and 66 kDa in column 3 represents Arg 1 sequence in the CD9-Arg1-in chimera.

FIG. 5C shows blot of 293f cells expressing a Arg1-CD9-out-tet construct (construct 1B of FIG. 1). Column 1 is molecular weight markers; column 2 is extracts from cells not treated with doxycycline and probed with anti-Arg1 antibody; column 3 is extracts from cells treated with doxycycline and probed with anti-Arg1 antibody. The band at approximately 42 kDa in columns 2 and 3 represents actin. The band above 66 kDa in column 3 represents Arg 1 sequence in the Arg1-CD9 chimera.

FIG. 5D shows blot of 293f cells expressing a CD9-Arg1-out-tet construct (construct 1C). Column 1 is molecular weight markers; column 2 is extracts from cells not treated with doxycycline and probed with anti-Arg1 antibody; column 3 is extracts from cells treated with doxycycline and probed with anti-Arg1 antibody. The band at approximately 42 kDa in columns 2 and 3 represents actin. The band above 66 kDa in column 3 represents Arg 1 sequence in the CD9-Arg1 chimera.

FIG. 6A is a histogram depicting numbers of exosome particles as determined by nanoparticle tracking analysis (NTA). From left to right, the first column represents exosomes produced from cells transduced with Arg1-tet (construct 1A); the second column represents exosomes produced from cells transduced with Arg1-CD9-out-tet (construct 1B); the third column represents exosomes produced from cells transduced with CD9-Arg1-out-tet (construct 1C); and the third column represents exosomes produced from cells transduced with CD9-Arg1-in-tet (construct 1D).

FIG. 6B is a histogram depicting protein content of exosomes as determined by Bicinchoninic Acid Protein Assay (BCA). From left to right, the first column represents exosomes produced from cells transduced with Arg1-tet (construct 1A); the second column represents exosomes produced from cells transduced with Arg1-CD9-out-tet (construct 1B); the third column represents exosomes produced from cells transduced with CD9-Arg1-out-tet (construct 1C); and the third column represents exosomes produced from cells transduced with CD9-Arg1-in-tet (construct 1D).

FIG. 6C is a histogram depicting Arg1 concentration of exosomes as determined by antibody assay. From left to right, the first column represents exosomes produced from cells transduced with Arg1-tet (construct 1A); the second column represents exosomes produced from cells transduced with Arg1-CD9-out-tet (construct 1B); the third column represents exosomes produced from cells transduced with CD9-Arg1-out-tet (construct 1C); and the third column represents exosomes produced from cells transduced with CD9-Arg1-in-tet (construct 1D).

FIG. 6D is a histogram depicting Arg1 enzymatic activity of exosomes as determined by urea production. From left to right, the first column represents exosomes produced from cells transduced with Arg1-tet (construct 1A); the second column represents exosomes produced from cells transduced with Arg1-CD9-out-tet (construct 1B); the third column represents exosomes produced from cells transduced with CD9-Arg1-out-tet (construct 1C); and the third column represents exosomes produced from cells transduced with CD9-Arg1-in-tet (construct 1D).

FIG. 7A is a histogram depicting Arg1 enzyme activity in U/L in 293F cells treated with 293F-derived exosomes as a function of Arg1 construct and time in hours after treatment. From left to right, column 1 depicts 293F cells treated with Arg1-free exosomes at 6-hours after treatment; column 2 depicts 293F cells treated with Arg1-free exosomes at 24-hours after treatment; column 3 depicts 293F cells treated with Arg1-free exosomes at 48-hours after treatment; column 4 depicts 293F cells treated with rArg1-loaded (construct 1A) exosomes at 6-hours after treatment; column 5 depicts 293F cells treated with rArg1-loaded (construct 1A) exosomes at 24-hours after treatment; column 6 depicts 293F cells treated with rArg1-loaded (construct 1A) exosomes at 48-hours after treatment; column 7 depicts 293F cells treated with 8.72 ng CD9-Arg1-in (construct 1D) exosomes at 6-hours after treatment; column 8 depicts 293F cells treated with 8.72 ng CD9-Arg1-in (construct 1D) exosomes at 24-hours after treatment; column 9 depicts 293F cells treated with 8.72 ng CD9-Arg1-in (construct 1D) exosomes at 48-hours after treatment. Here, 293F cells treated with 8.72 ng rArg1 showed similar levels of Arg1 activity in the cells compared with 293F exosomes without rArg1 construct. Increased Arg1 activities were detected in 293F cells treated with CD9-Arg1-in exosomes 6 hours after treatment in a time dependent manner.

FIG. 7B is a histogram depicting Arg1 enzyme activity in U/L in HepG2 cells treated with 293F-derived exosomes as a function of Arg1 construct and time in hours after treatment. From left to right, column 1 depicts HepG2 cells treated with Arg1-free exosomes at 6-hours after treatment; column 2 depicts HepG2 cells treated with Arg1-free exosomes at 24-hours after treatment; column 3 depicts HepG2 cells treated with Arg1-free exosomes at 48-hours after treatment; column 4 depicts HepG2 cells treated with rArg1-loaded (construct 1A) exosomes at 6-hours after treatment; column 5 depicts HepG2 cells treated with rArg1-loaded (construct 1A) exosomes at 24-hours after treatment; column 6 depicts HepG2 cells treated with rArg1-loaded (construct 1A) exosomes at 48-hours after treatment; column 7 depicts HepG2 cells treated with 8.72 ng CD9-Arg1-in (construct 1D) exosomes at 6-hours after treatment; column 8 depicts HepG2 cells treated with 8.72 ng CD9-Arg1-in (construct 1D) exosomes at 24-hours after treatment; column 9 depicts HepG2 cells treated with 8.72 ng CD9-Arg1-in (construct 1D) exosomes at 48-hours after treatment. Here, HepG2 cells treated with 8.72 ng rArg1 showed similar levels of Arg1 activity in the cells compared with 293F exosomes without rArg1 construct. Increased Arg1 activities were detected in HepG2 cells treated with CD9-Arg1-in exosomes 6 hours after treatment in a time dependent manner.

FIG. 7C is a histogram depicting urea production in mg/dL in 293F cells treated with 293F-derived exosomes as a function of Arg1 construct and time in hours after treatment. From left to right, column 1 depicts 293F cells treated with Arg1-free exosomes at 6-hours after treatment; column 2 depicts 293F cells treated with Arg1-free exosomes at 24-hours after treatment; column 3 depicts 293F cells treated with Arg1-free exosomes at 48-hours after treatment; column 4 depicts 293F cells treated with rArg1-loaded (construct 1A) exosomes at 6-hours after treatment; column 5 depicts 293F cells treated with rArg1-loaded (construct 1A) exosomes at 24-hours after treatment; column 6 depicts 293F cells treated with rArg1-loaded (construct 1A) exosomes at 48-hours after treatment; column 7 depicts 293F cells treated with 8.72 ng CD9-Arg1-in (construct 1D) exosomes at 6-hours after treatment; column 8 depicts 293F cells treated with 8.72 ng CD9-Arg1-in (construct 1D) exosomes at 24-hours after treatment; column 9 depicts 293F cells treated with 8.72 ng CD9-Arg1-in (construct 1D) exosomes at 48-hours after treatment. Here, 293F cells treated with 8.72 ng rArg1 showed similar levels of urea production in the cells compared with 293F exosomes without rArg1 construct. Increased urea production was detected in 293F cells treated with CD9-Arg1-in exosomes 6 hours after treatment in a time dependent manner.

FIG. 7D is a histogram depicting urea production in mg/dL in HepG2 cells treated with 293F-derived exosomes as a function of Arg1 construct and time in hours after treatment. From left to right, column 1 depicts HepG2 cells treated with Arg1-free exosomes at 6-hours after treatment; column 2 depicts HepG2 cells treated with Arg1-free exosomes at 24-hours after treatment; column 3 depicts HepG2 cells treated with Arg1-free exosomes at 48-hours after treatment; column 4 depicts HepG2 cells treated with rArg1-loaded (construct 1A) exosomes at 6-hours after treatment; column 5 depicts HepG2 cells treated with rArg1-loaded (construct 1A) exosomes at 24-hours after treatment; column 6 depicts HepG2 cells treated with rArg1-loaded (construct 1A) exosomes at 48-hours after treatment; column 7 depicts HepG2 cells treated with 8.72 ng CD9-Arg1-in (construct 1D) exosomes at 6-hours after treatment; column 8 depicts HepG2 cells treated with 8.72 ng CD9-Arg1-in (construct 1D) exosomes at 24-hours after treatment; column 9 depicts HepG2 cells treated with 8.72 ng CD9-Arg1-in (construct 1D) exosomes at 48-hours after treatment. Here, HepG2 cells treated with 8.72 ng rArg1 showed similar levels of urea production in the cells compared with 293F exosomes without rArg1 construct. Increased urea production was detected in HepG2 cells treated with CD9-Arg1-in exosomes 6 hours after treatment in a time dependent manner.

FIG. 8A is a histogram depicting net increase in Arg1 enzyme activity in U/L in HepG2 cells treated with rArg1 as a function of time and dosage. From left to right, bar 1=6 hours after treating HepG2 cells with 1250 ng rArg1, bar 2=24 hours after treating with 1250 ng rArg1, bar 3=48 hours after treating with 1250 ng rArg1, bar 4=6 hours after treating HepG2 cells with 250 ng rArg1, bar 5=24 hours after treating with 8250 ng rArg1, bar 6=48 hours after treating with 250 ng rArg1, bar 7=6 hours after treating HepG2 cells with 50 ng rArg1, bar 8=24 hours after treating with 50 ng rArg1, bar 9=48 hours after treating with 50 ng rArg1, bar 10=6 hours after treating HepG2 cells with 8.72 ng rArg1, bar 11=24 hours after treating with 8.72 ng rArg1, and bar 12=48 hours after treating with 8.72 ng rArg1.

FIG. 8B is a histogram depicting net increase in Arg1 enzyme activity in U/L in HepG2 cells treated with CD9-Arg1-in-containing exosomes as a function of time and dosage. From left to right, bar 1=6 hours after treating HepG2 cells with exosomes containing 250 ng CD9-Arg1-in, bar 2=24 hours after treating HepG2 cells with exosomes containing 250 ng CD9-Arg1-in, bar 3=48 hours after treating HepG2 cells with exosomes containing 250 ng CD9-Arg1-in, bar 4=6 hours after treating HepG2 cells with exosomes containing 50 ng CD9-Arg1-in, bar 5=24 hours after treating HepG2 cells with exosomes containing 50 ng CD9-Arg1-in, bar 6=48 hours after treating HepG2 cells with exosomes containing 50 ng CD9-Arg1-in, bar 7=6 hours after treating HepG2 cells with exosomes containing 8.72 ng CD9-Arg1-in, bar 8=24 hours after treating HepG2 cells with exosomes containing 8.72 ng CD9-Arg1-in, bar 9=48 hours after treating HepG2 cells with exosomes containing 8.72 ng CD9-Arg1-in. Here, CD9-Arg-in exosomes containing nanogram levels of Arg1 (˜8.72 ng Arg1 per 1E11 CD9-Arg1-in exosome particles) showed high arginase activity similar to that of microgram levels of recombinant Arg1 protein. The delivery of Arg1 into HepG2 cells via CD9-Arg1-in exosomes showed both time and dose-dependent effect. Furthermore, CD9-Arg1-in exosomes showed higher potency in catalyzing arginine compared to recombinant Arg1.

FIG. 8C is a histogram depicting urea production in mg/dL in HepG2 cells treated with rArg1 as a function of time and dosage. From left to right, bar 1=6 hours after treating HepG2 cells with 1250 ng rArg1, bar 2=24 hours after treating with 1250 ng rArg1, bar 3=48 hours after treating with 1250 ng rArg1, bar 4=6 hours after treating HepG2 cells with 250 ng rArg1, bar 5=24 hours after treating with 8250 ng rArg1, bar 6=48 hours after treating with 250 ng rArg1, bar 7=6 hours after treating HepG2 cells with 50 ng rArg1, bar 8=24 hours after treating with 50 ng rArg1, bar 9=48 hours after treating with 50 ng rArg1, bar 10=6 hours after treating HepG2 cells with 8.72 ng rArg1, bar 11=24 hours after treating with 8.72 ng rArg1, and bar 12=48 hours after treating with 8.72 ng rArg1.

FIG. 8D is a histogram depicting urea production in mg/dL in HepG2 cells treated with CD9-Arg1-in-containing exosomes as a function of time and dosage. From left to right, bar 1=6 hours after treating HepG2 cells with exosomes containing 250 ng CD9-Arg1-in, bar 2=24 hours after treating HepG2 cells with exosomes containing 250 ng CD9-Arg1-in, bar 3=48 hours after treating HepG2 cells with exosomes containing 250 ng CD9-Arg1-in, bar 4=6 hours after treating HepG2 cells with exosomes containing 50 ng CD9-Arg1-in, bar 5=24 hours after treating HepG2 cells with exosomes containing 50 ng CD9-Arg1-in, bar 6=48 hours after treating HepG2 cells with exosomes containing 50 ng CD9-Arg1-in, bar 7=6 hours after treating HepG2 cells with exosomes containing 8.72 ng CD9-Arg1-in, bar 8=24 hours after treating HepG2 cells with exosomes containing 8.72 ng CD9-Arg1-in, bar 9=48 hours after treating HepG2 cells with exosomes containing 8.72 ng CD9-Arg1-in. Here, CD9-Arg-in exosomes containing nanogram levels of Arg1 (˜8.72 ng Arg1 per 1E11 CD9-Arg1-in exosome particles) showed efficacy in catalyzing arginine into urea similar to that of microgram levels of recombinant Arg1 protein. The delivery of Arg1 into HepG2 cells via CD9-Arg1-in exosomes showed both time and dose-dependent effect. Furthermore, CD9-Arg1-in exosomes showed higher potency in producing urea compared to recombinant Arg1. Here, 1E11 CD9-Arg1-in exosomes containing 8.72 ng Arg1 showed similar urea production with 1250 ng Arg1.

FIG. 9A is a histogram depicting Arg1 enzyme activity in HepG2 cells (U/L) as a function of time and Arg1 construct delivered by exosomes. From left to right, bar 1=6 hours after treating HepG2 cells with 1E11 exosomes (8.72 ng Arg1 per 1E11 exosomes) containing the CD9-Arg1-in construct, bar 2=24 hours after treating with 1E11 exosomes (8.72 ng Arg1 per 1E11 exosomes) containing the CD9-Arg1-in construct, bar 3=48 hours after treating with 1E11 exosomes (8.72 ng Arg1 per 1E11 exosomes) containing the CD9-Arg1-in construct, bar 4=6 hours after treating HepG2 cells with 1.96E11 exosomes containing the Arg1-CD9-out-tet construct, bar 5=24 hours after treating with 1.96E11 exosomes containing the Arg1-CD9-out-tet construct, bar 6=48 hours after treating with 1.96E11 exosomes containing the Arg1-CD9-out-tet construct, bar 7=6 hours after treating HepG2 cells with 1E11 exosomes containing the Arg1-CD9-out-tet construct, bar 8=24 hours after treating with 1E11 exosomes containing the Arg1-CD9-out-tet construct, and bar 9=48 hours after treating with 1E11 exosomes containing the Arg1-CD9-out-tet construct.

FIG. 9B is a histogram depicting urea production in HepG2 cells (mg/dL) as a function of time and Arg1 construct delivered by exosomes. From left to right, bar 1=6 hours after treating HepG2 cells with 1E11 exosomes (8.72 ng Arg1 per 1E11 exosomes) containing the CD9-Arg1-in construct, bar 2=24 hours after treating with 1E11 exosomes (8.72 ng Arg1 per 1E11 exosomes) containing the CD9-Arg1-in construct, bar 3=48 hours after treating with 1E11 exosomes (8.72 ng Arg1 per 1E11 exosomes) containing the CD9-Arg1-in construct, bar 4=6 hours after treating HepG2 cells with 1.96E11 exosomes containing the Arg1-CD9-out-tet construct, bar 5=24 hours after treating with 1.96E11 exosomes containing the Arg1-CD9-out-tet construct, bar 6=48 hours after treating with 1.96E11 exosomes containing the Arg1-CD9-out-tet construct, bar 7=6 hours after treating HepG2 cells with 1E11 exosomes containing the Arg1-CD9-out-tet construct, bar 8=24 hours after treating with 1E11 exosomes containing the Arg1-CD9-out-tet construct, and bar 9=48 hours after treating with 1E11 exosomes containing the Arg1-CD9-out-tet construct. Here, exosomes carrying extracellular N-terminal Arg1 (construct 1B) were more efficient in catalyzing arginine and producing urea, with ˜50% less of Arg1 carried by Arg1-CD9-out-tet exosomes, urea concentrations in the culture supernatants were similar to CD9-Arg1-in exosomes. However, despite the higher efficiency in producing urea, Arg1-CD9-out-tet exosomes failed to deliver Arg1 into HepG2 cells.

FIG. 10 is a histogram depicting urea concentration in media (mg/dL) as a function of time, and Arg1 construct and amount. CD9-Arg1-in-containing exosomes were compared to Arg-CD9-out-tet-containing exosomes and Arg1 for catalyzing the production of urea in cell culture. From left to right, the first bar (bar 1) represents 6 hours after treating with CD9-Arg1-in-containing exosomes, bar 2=24 hours after treating with CD9-Arg1-in-containing exosomes, bar 3=48 hours after treating with CD9-Arg1-in-containing exosomes, bar 4=6 hours after treating with Arg1-CD9-out-tet-containing exosomes, bar 5=24 hours after treating with Arg1-CD9-out-tet-containing exosomes, bar 6=48 hours after treating with Arg1-CD9-out-tet-containing exosomes, bar 7=6 hours after treating with 1250 ng rArg1, bar 8=24 hours after treating with 1250 ng rArg1, bar 9=48 hours after treating with 1250 ng rArg1, bar 10=6 hours after treating with 250 ng rArg1, bar 11=24 hours after treating with 250 ng rArg1, bar 12=48 hours after treating with 250 ng rArg1, bar 13=6 hours after treating with 50 ng rArg1, bar 14=24 hours after treating with 50 ng rArg1, bar 15=48 hours after treating with 50 ng rArg1, bar 16=6 hours after treating with 8.72 ng rArg1, bar 17=24 hours after treating with 8.72 ng rArg1, and bar 18=48 hours after treating with 8.72 ng rArg1. Here it was observed that nanogram levels of Arg1-loaded exosomes effectively catalyzed arginine into urea similar to microgram levels of recombinant Arg1 protein (rArg1). Exosomes containing an extracellular Arg1 construct (Arg1-CD9-out-tet [construct 1B]) were more potent in catalyzing arginine, whereas exosomes containing an intracellular Arg1 construct (CD9-Arg1-in [construct 1D]) were able to deliver the functional Arg1 into the cells. Here, urea production by recombinant Arg1 took place outside of the cells. The incapability of Arg1 to enter the hepatocytes in vivo is known to limit the therapeutic efficacy in Arg1 deficiency, therefore it is envisioned that intracellular Arg1 activity in hepatocytes, as shown here with the CD9-Arg1-in construct-containing exosomes, is important for drug efficacy.

FIG. 11 is a histogram depicting Arg1 enzyme activity in HepG2 cells (U/L) as a function of time (6 hours, 24 hours, 48 hours) and Arg1 construct (recombinant human Arg1, and 1E11 293F exosomes loaded with 8.7 ng Arginase as CD9-Arg1-in (construct 1D). Here, exosomes carrying intracellular N-terminal Arg1 (construct 1D) were more efficient in catalyzing arginine and producing urea with nanogram amounts of arginase (8.7 ng) than milligram amounts of exosomeless recombinant arginase 1. From left to right, bar 1=6 hours after treating HepG2 cells with 1E11 CD9-Arg-in construct (8.7 ng CD9-Arg-in), bar 2=24 hours after treating with 8.7 ng CD9-Arg-in, bar 3=48 hours after treating with 8.7 ng CD9-Arg-in, bar 4=6 hours after treating HepG2 cells with 8.72 ng rArg1, bar 5=24 hours after treating with 8.72 ng rArg1, bar 6=48 hours after treating with 8.72 ng rArg1, bar 7=6 hours after treating HepG2 cells with 50 ng rArg1, bar 8=24 hours after treating with 50 ng rArg1, bar 9=48 hours after treating with 50 ng rArg1, bar 10=6 hours after treating HepG2 cells with 250 ng rArg1, bar 11=24 hours after treating with 250 ng rArg1, and bar 12=48 hours after treating with 250 ng rArg1, bar 13=6 hours after treating HepG2 cells with 1,250 ng rArg1, bar 14=24 hours after treating with 1,250 ng rArg1, and bar 15=48 hours after treating with 1,250 ng rArg1.

FIG. 12A is a histogram depicting the amount of arginase 1 in picograms per tissue (delivered as CD9-Arg-in exosomes) in the liver of Arg1 knockout mice as a function of time or dose. From left to right, bar 1=wildtype mice without exogenous CD9-Arg-in showing wildtype levels of arginase 1; bar 2=Arg1 KO mice immediately after receiving exogenous CD9-Arg-in showing less-than wildtype levels of arginase 1; bar 3=Arg1 KO mice 2 hours after receiving exogenous CD9-Arg-in showing wildtype levels of arginase 1; bar 4=Arg1 KO mice 24 hours after receiving exogenous CD9-Arg-in showing a drop from wildtype levels of arginase 1; bar 5 Arg1 KO mice after receiving 2 doses of exogenous CD9-Arg-in showing an increase in Arg1 levels after slight drop shown in bar 4.

FIG. 12B is a dot plot depicting the amount of arginine in micromoles per liter detected in the plasma of Arg1 knockout mice as a function of time or dose of exogenous exosomes harboring CD9-Arg-in (CD9-Arg-in). From left to right, the first column depicts plasma arginine levels in wildtype mice; the second column depicts plasma arginine levels in Arg1 knock-out mice immediately after receiving CD9-Arg-in; the third column depicts plasma arginine levels in Arg1 knock-out mice 2 hours after receiving CD9-Arg-in; the fourth column depicts plasma arginine levels in Arg1 knock-out mice 6 hours after receiving CD9-Arg-in; and the fifth column depicts plasma arginine levels in Arg1 knock-out mice 24 hours after receiving CD9-Arg-in.

FIG. 13 is a line graph depicting the percent survival of Arg1 knock out mice as a function of Arg1 administration over time by day. The black line, which depicts 0% survival at day 13, represents PBS (no Arg1 dosage) administered mice. The green and red lines, which depict about 10% and 25% survival at day 18, respectively, depict mice administered in two studies with exosomes harboring CD9-Arg-in construct.

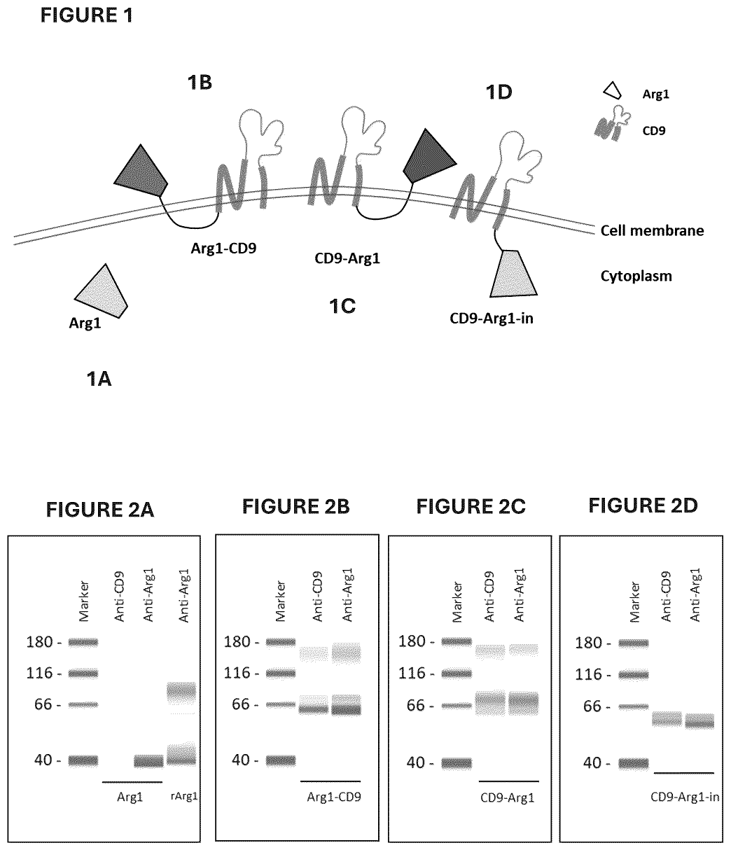

### DETAILED DESCRIPTION OF EMBODIMENTS

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, “and/or” refers to and encompasses any and all possible combinations of one or more of the associated listed items, as well as the lack of combinations when interpreted in the alternative (or).

The terms “about” and “approximate”, as used herein when referring to a measurable value such as an amount, dose, time, temperature, activity, level, number, frequency, percentage, dimension, size, amount, weight, position, length and the like, is meant to encompass variations of ±15%, ±10%, ±5%, ±1%, ±0.5%, or even ±0.1% of the specified amount, dose, time, temperature, activity, level, number, frequency, percentage, dimension, size, amount, weight, position, length and the like. In instances in which the terms “about” and “approximate” are used in connection with the location or position of regions within a reference polypeptide, these terms encompass variations of ± up to 20 amino acid residues, ± up to 15 amino acid residues, ± up to 10 amino acid residues, ± up to 5 amino acid residues, ± up to 4 amino acid residues, ± up to 3 amino acid residues, ± up to 2 amino acid residues, or even ±1 amino acid residue.

The term “derived from” as in “A is derived from B” means that A is obtained from B in such a manner that A is not identical to B.

The terms “treat”, “therapeutic”, “prophylactic” and “prevent” are not intended to be absolute terms. Treatment, prevention and prophylaxis can refer to any delay in onset, amelioration of symptoms, improvement in patient survival, increase in survival time or rate, etc. Treatment, prevention, and prophylaxis can be complete or partial. The term “prophylactic” means not only “prevent”, but also minimize illness and disease. For example, a “prophylactic” agent can be administered to a subject, e.g., a human subject, to prevent infection, or to minimize the extent of illness and disease caused by such infection. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects, the severity of disease is reduced by at least 25%, 50%, 75%, 80%, or 90%, or in some cases, no longer detectable using standard diagnostic techniques.

A treatment can be considered “effective,” as used herein, if one or more of the signs or symptoms of a condition described herein are altered in a beneficial manner, other clinically accepted symptoms are improved, or even ameliorated, or a desired response is induced e.g., by at least 2%, 3%, 4%, 5%, 10%, or more, following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (e.g., progression of the disease is halted). Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. One skilled in the art can monitor efficacy of administration and/or treatment by measuring any one of such parameters, or any combination of parameters.

The term “effective amount” as used herein refers to the amount of a composition or an agent needed to alleviate at least one or more symptom of the disease or disorder, and relates to a sufficient amount of therapeutic composition to provide the desired effect. The term “therapeutically effective amount” refers to an amount of a composition or therapeutic agent that is sufficient to provide a particular effect when administered to a typical subject. An effective amount as used herein, in various contexts, can include an amount sufficient to delay the development of a symptom of the disease, alter the course of a symptom disease (for example but not limited to, slowing the progression of a symptom of the disease), or reverse a symptom of the disease. For example, for the given parameter, a therapeutically effective amount will show an increase or decrease of therapeutic effect at least any of 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least any of a 1.2-fold, 1.5-fold, 2-fold, 5-fold, or more effect over a control. The therapeutically effective amount may be administered in one or more doses of the therapeutic agent. The therapeutically effective amount may be administered in a single administration, or over a period of time in a plurality of doses.

“Administering” as used herein can include any suitable routes of administering a therapeutic agent or composition as disclosed herein. Suitable routes of administration include, without limitation, oral, parenteral, intravenous, intramuscular, subcutaneous, transdermal, airway (aerosol), pulmonary, cutaneous, injection or topical administration. Administration can be local or systemic.

As used herein, the term “pharmaceutically acceptable” refers to a carrier that is compatible with the other ingredients of the formulation and not deleterious to the recipient thereof. The term is used synonymously with “physiologically acceptable” and “pharmacologically acceptable”. A pharmaceutical composition will generally comprise agents for buffering and preservation in storage, and can include buffers and carriers for appropriate delivery, depending on the route of administration. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “dose” and “dosage” are used interchangeably herein. A dose refers to the amount of active ingredient given to an individual at each administration. For the present invention, the dose can refer to the concentration of the extracellular vesicles or associated components, e.g., the amount of therapeutic agent or dosage of radiolabel. The dose will vary depending on a number of factors, including frequency of administration; size and tolerance of the individual; severity of the condition; risk of side effects; the route of administration; and the imaging modality of the detectable moiety (if present). One of skill in the art will recognize that the dose can be modified depending on the above factors or based on therapeutic progress. The term “dosage form” refers to the particular format of the pharmaceutical and depends on the route of administration. For example, a dosage form can be in a liquid, e.g., a saline solution for injection.

“Subject,” “patient,” “individual” and like terms are used interchangeably and refer to, except where indicated, mammals such as humans and non-human primates, as well as rabbits, rats, mice, goats, pigs, and other mammalian species. The term does not necessarily indicate that the subject has been diagnosed with a particular disease, but typically refers to an individual under medical supervision. A patient can be an individual that is seeking treatment, monitoring, adjustment or modification of an existing therapeutic regimen, etc.

The terms “loaded,” “loaded with,” “is/are loaded with,” and similar terms referring to the coupling or association of a molecular entity with an extracellular vesicle (e.g., exosome) mean any physical association by any force of any one or more molecular entities with an exosome. The terms include without limitation, (i) physical association of a molecular entity at, on, or proximate the outer membrane of the extracellular vesicle such that the molecular entity is positioned outside the extracellular vesicle, (ii) physical association of a molecular entity at, on, or proximate the inner membrane of the extracellular vesicle such that the molecular entity is positioned inside the extracellular vesicle, (iii) physical association of a molecular entity partially or fully within the membrane of the extracellular vesicle, (iv) positioning of the molecular entity within the lumen of the extracellular vesicle, and (v) combinations thereof. In some cases, in which the molecular entity is partially within the membrane, the molecular entity may be positioned such that, when the molecular entity is or comprises a polypeptide, the carboxyterminal end of the entity protrudes into the lumen of the extracellular vesicle, the carboxyterminal end of the entity protrudes outside of the extracellular vesicle, the carboxyterminal end of the entity protrudes into the lumen of the extracellular vesicle and the aminoterminal end of the entity protrudes outside the extracellular vesicle, the aminoterminal end of the entity protrudes into the lumen of the extracellular vesicle and the carboxyterminal end of the entity protrudes outside the extracellular vesicle, the aminoterminal end of the entity protrudes into the lumen of the extracellular vesicle, or the aminoterminal end of the entity protrudes outside the extracellular vesicle.

Here, “molecular entity” includes without limitation polypeptides, chimeric polypeptides, active sites or working end of proteins, parts of polypeptides, other biological molecules, small organics molecules, synthetic molecules, and the like.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The phrase “at least one” includes “a plurality”.

Definitions of common terms in cell biology and molecular biology can be found in “The Merck Manual of Diagnosis and Therapy”, 19th Edition, published by Merck Research Laboratories, 2006 (ISBN 0-91 1910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10:0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

The term “native form” corresponds to the polypeptide as it is understood to be encoded by the infectious agent's genome. The term “exosomal form” corresponds to any derivative of the protein that, in whole or in part, is fused to an exosome-associated protein. The term “cytoplasmic form” corresponds to any derivative of the protein that, in whole or in part, is configured, or designed, to be expressed within the cytoplasm of the cell, rather than entering the canonical secretory pathway.

The expression that a certain protein is “configured, or designed, to be expressed” in a certain way means that its nucleotide sequence encodes certain a particular amino acid sequence such that when that protein is expressed in a cell, that protein will be in its native form, exosomal form, or cytoplasmic form by virtue of that particular amino acid sequence. For instance, if a spike protein(S) is expressed in its native form, it is configured, or designed, to induce a humoral or cellular immune response by virtue of the fact that it is a transmembrane protein with an extracellular domain.

The term “extracellular vesicle” (EV) refers to lipid bilayer-delimited particles that are naturally released from cells. EVs range in diameter from around 20-30 nanometers to about 10 microns or more. EVs can comprise proteins, nucleic acids, lipids and metabolites from the cells that produced them. EVs include exosomes (about 50 to about 200 nm), microvesicles (about 100 to about 300 nm), ectosomes (about 50 to about 1000 nm), apoptotic bodies (about 50 to about 5000 nm) and lipid-protein aggregates of the same dimensions.

A variety of host cells are known in the art and suitable for proteins expression and extracellular vesicles production. Non-limiting examples of typical cell used for transfection include, but are not limited to, a bacterial cell, a eukaryotic cell, a yeast cell, an insect cell, or a plant cell. For example, human embryonic kidney 293 (HEK293), E. coli, Bacillus, Streptomyces, Pichia pastoris, Salmonella typhimurium, Drosophila S2, Spodoptera SJ9, CHO, COS (e.g. COS-7), 3T3-F442A, HeLa, HUVEC, HUAEC, NIH 3T3, Jurkat, 293, 293H, or 293F. See, e.g., Portolano et al., “Recombinant Protein Expression for Structural Biology in HEK 293F Suspension Cells: A Novel and Accessible Approach,” Journal of Visualized Experiments, October 2014, 92, e51897, pp. 1-8 for a description of the recombinant proteins in 293 cells in suspension culture.

Extracellular vesicles (EVs) are lipid bound vesicles secreted by cells into the extracellular space. The three main subtypes of EVs are microvesicles (MVs), exosomes, and apoptotic bodies, which are differentiated based upon their biogenesis, release pathways, size, content, and function. For a review of extracellular vesicles, see, e.g., Doyle and Wang, “Overview of Extracellular Vesicles, Their Origin, Composition, Purpose, and Methods for Exosome Isolation and Analysis,” Cells, v.8 (7), 2019 July, and references therein.

Exosomes include small, secreted vesicles of about 20-200 nm in diameter that are released by inter alia mammalian cells, and made either by budding into endosomes or by budding from the plasma membrane of a cell. In some cases, exosomes have a characteristic buoyant density of approximately 1.1-1.2 g/mL, and a characteristic lipid composition. Their lipid membrane is typically rich in cholesterol and contains sphingomyelin, ceramide, lipid rafts and exposed phosphatidylserine. Exosomes express certain marker proteins, such as integrins and cell adhesion molecules, but generally lack markers of lysosomes, mitochondria, or caveolae. In some embodiments, the exosomes contain cell-derived components, such as, but not limited to, proteins, DNA and RNA (e.g., microRNA [miR] and noncoding RNA). In some embodiments, exosomes can be obtained from cells obtained from a source that is allogeneic, autologous, xenogeneic, or syngeneic with respect to the recipient of the exosomes.

Certain types of RNA, e.g., microRNA (miRNA), are known to be carried by exosomes. miRNAs function as post-transcriptional regulators, often through binding to complementary sequences on target messenger RNA transcripts (mRNAs), thereby resulting in translational repression, target mRNA degradation and/or gene silencing.

Useful exosomes can be obtained from any cell source, including prokaryotes, plants, fungi, metazoans, vertebrate, mammalian, primate, human, autologous cells and allogeneic cells. See, e.g., Kim et al., “Platform technologies and human cell lines for the production of therapeutic exosomes,” Extracell Vesicles Circ Nucleic Acids 2021; 2:3-17. For example, exosomes may be derived from mesenchymal stem cells, embryonic stem cells, iPS cells, immune cells, PBMCs, neural stem cells, HEK293 cells, which are described in e.g., Dumont et al., “Human cell lines for biopharmaceutical manufacturing: history, status, and future perspectives,” Crit Rev Biotechnol 2016; 36:1110-22, HEK293T cells, which are described in e.g., Li et al., “Identification and characterization of 293T cell-derived exosomes by profiling the protein, mRNA and MicroRNA components,” PLOS One 2016; 11: e0163043, 293F cells, Stenkamp et al., “Exosomes represent a novel mechanism of regulatory T cell suppression (P1079),” J Immunol May 1, 2013, 190 (1 Supplement) 121.11, amniotic cells, CAR-T cells, cardiospheres and cardiosphere-derived cells (CDCs), which are described in, e.g., WO2014028493, WO2022006178A1, US20210032598A1, U.S. Pat. No. 9,828,603B2, EP2914273A1, US20200316226A1, US20120315252A1, US20170360842A1, and references therein, and the like.

Briefly, methods for preparing exosomes can include the steps of: culturing cells in media, isolating the cells from the media, purifying the exosome by, e.g., sequential centrifugation, and optionally, clarifying the exosomes on a density gradient, e.g., sucrose density gradient. In some instances, the isolated and purified exosomes are essentially free of non-exosome components, such as cellular components or whole cells. Exosomes can be resuspended in a buffer such as a sterile PBS buffer containing 0.01-1% human serum albumin. The exosomes may be frozen and stored for future use.

Exosomes can be collected, concentrated and/or purified using methods known in the art. For example, differential centrifugation has become a leading technique wherein secreted exosomes are isolated from the supernatants of cultured cells. This approach allows for separation of exosomes from larger extracellular vesicles and from most non-particulate contaminants by exploiting their size. Exosomes can be prepared as described in a wide array of papers, including but not limited to, Fordjour et al., “A shared pathway of exosome biogenesis operates at plasma and endosome membranes”, bioRxiv, preprint posted Feb. 11, 2019, at https://www.biorxiv.org/content/10.1101/545228v1; Booth et ah, “Exosomes and HIV Gag bud from endosome-like domains of the T cell plasma membrane”, J Cell Biol., 172:923-935 (2006); and, Fang et ah, “Higher-order oligomerization targets plasma membrane proteins and HIV gag to exosomes”, PLOS Biol., 5: el58 (2007). Exosomes using a commercial kit such as, but not limited to the ExoSpin™ Exosome Purification Kit, Invitrogen® Total Exosome Purification Kit, PureExo® Exosome Isolation Kit, and ExoCap™ Exosome Isolation kit. Methods for isolating exosome from stem cells are found in, e.g., Tan et ah, Journal of Extracellular Vesicles, 2:22614 (2013); Ono et ah, Sci Signal, 7 (332): ra63 (2014) and U.S. Application Publication Nos. 2012/0093885 and 2014/0004601. Methods for isolating exosome from cardiosphere-derived cells are found in, e.g., Ibrahim et al., “Exosomes as critical agents of cardiac regeneration triggered by cell therapy,” Stem Cell Reports, 2014. Specific methodologies include ultracentrifugation, density gradient, HPLC, adherence to substrate based on affinity, or filtration based on size exclusion.

Size exclusion allows for their separation from biochemically similar, but biophysically different microvesicles, which possess larger diameters of up to 1,000 nm. Differences in flotation velocity further allows for separation of differentially sized exosomes. In general, exosome sizes will possess a diameter ranging from 30-200 nm, including sizes of 40-100 nm. Further purification may rely on specific properties of the particular exosomes of interest. This includes, e.g., use of immunoadsorption with a protein of interest to select specific vesicles with exoplasmic or outward orientations.

Among current methods, e.g., differential centrifugation, discontinuous density gradients, immunoaffinity, ultrafiltration and high-performance liquid chromatography (HPLC), differential ultracentrifugation is the most commonly used for exosome isolation. This technique utilizes increasing centrifugal force from 2,000×g to 10,000×g to separate the medium- and larger-sized particles and cell debris from the exosome pellet at 100,000×g. Centrifugation alone allows for significant separation/collection of exosomes from a conditioned medium, although it may be insufficient to remove various protein aggregates, genetic materials, particulates from media and cell debris that are common contaminants. Enhanced specificity of exosome purification may deploy sequential centrifugation in combination with ultrafiltration, or equilibrium density gradient centrifugation in a sucrose density gradient, to provide for the greater purity of the exosome preparation (flotation density 1.1-1.2 g/mL) or application of a discrete sugar cushion in preparation.

Ultrafiltration can be used to purify exosomes without compromising their biological activity. Membranes with different pore sizes-such as 100 kDa molecular weight cutoff (MWCO) and gel filtration to eliminate smaller particles—have been used to avoid the use of a nonneutral pH or non-physiological salt concentration. Currently available tangential flow filtration (TFF) systems are scalable (to >10,000 L), allowing one to not only purify, but concentrate the exosome fractions, and such approaches are less time consuming than differential centrifugation. HPLC can also be used to purify exosomes to more uniformly sized particle preparations and preserve their biological activity as the preparation is maintained at a physiological pH and salt concentration. Other chemical methods have exploit differential solubility of exosomes for precipitation techniques, addition to volume-excluding polymers (e.g., polyethylene glycols (PEGs)), possibly combined additional rounds of centrifugation or filtration. For example, a precipitation reagent, ExoQuick®, can be added to conditioned cell media to quickly and rapidly precipitate a population of exosomes, although re-suspension of pellets prepared via this technique may be difficult. Flow field-flow fractionation (FIFFF) is an elution-based technique that is used to separate and characterize macromolecules (e.g., proteins) and nano-to micro-sized particles (e.g., organelles and cells) and which has been successfully applied to fractionate exosomes from culture media.

Beyond these techniques relying on general biochemical and biophysical features, focused techniques may be applied to isolate specific exosomes of interest. This includes relying on antibody immunoaffinity to recognizing certain exosome-associated antigens. As described, exosomes further express the extracellular domain of membrane-bound receptors at the surface of the membrane. This presents an opportunity for isolating and segregating exosomes in connection with their parental cellular origin, based on a shared antigenic profile. Conjugation to magnetic beads (e.g., such as anti-CD81 magnetic beads), chromatography matrices, plates or microfluidic devices allows isolating of specific exosome populations of interest as may be related to their production from a parent cell of interest or associated cellular regulatory state. Other affinity-capture methods use lectins which bind to specific saccharide residues on the exosome surface.

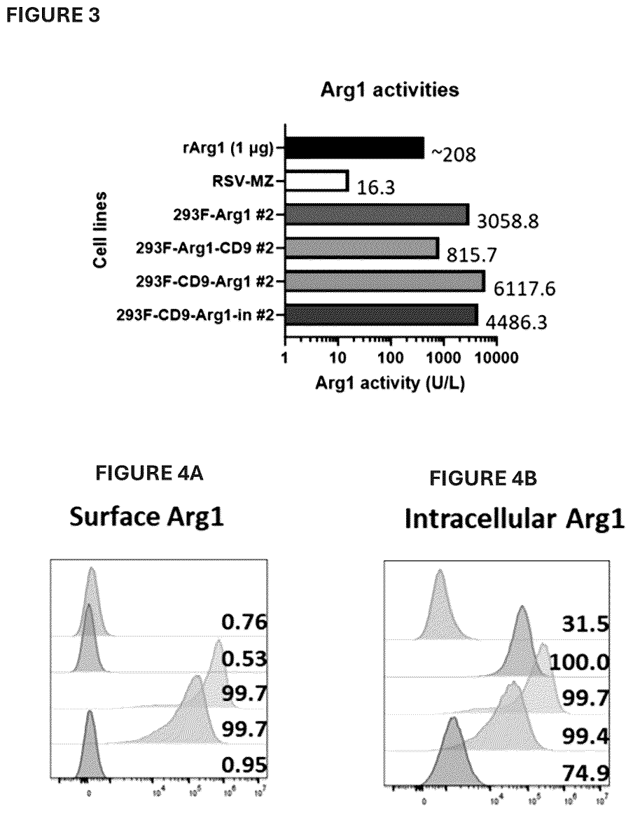

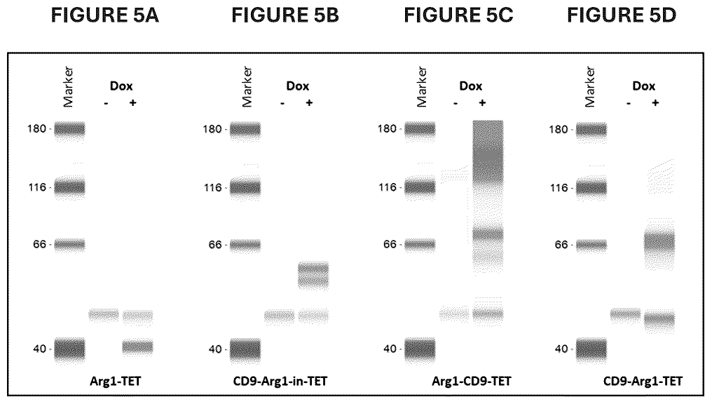

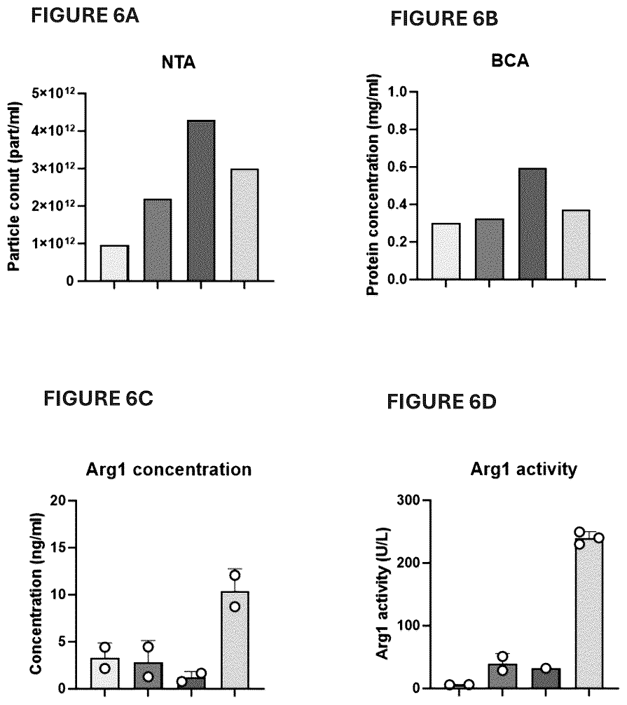

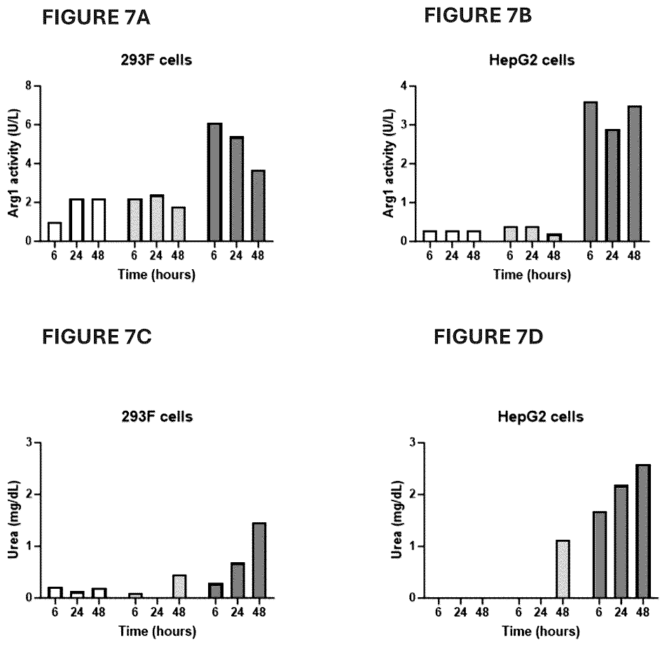

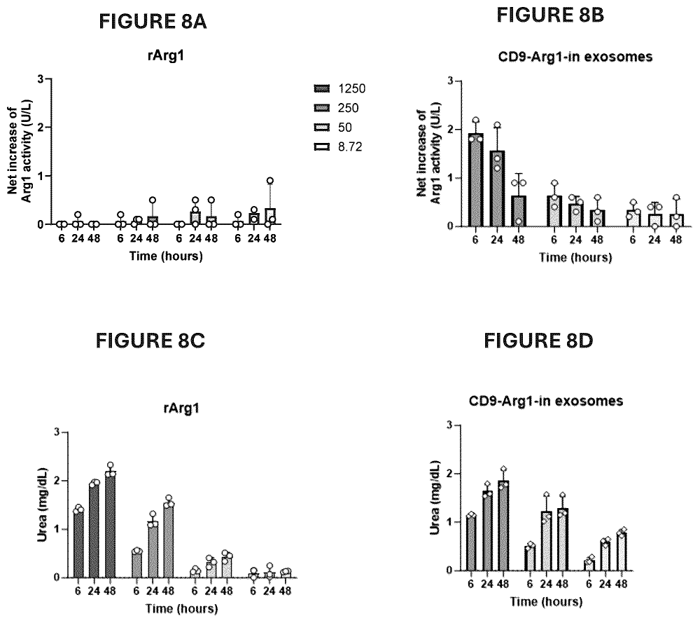

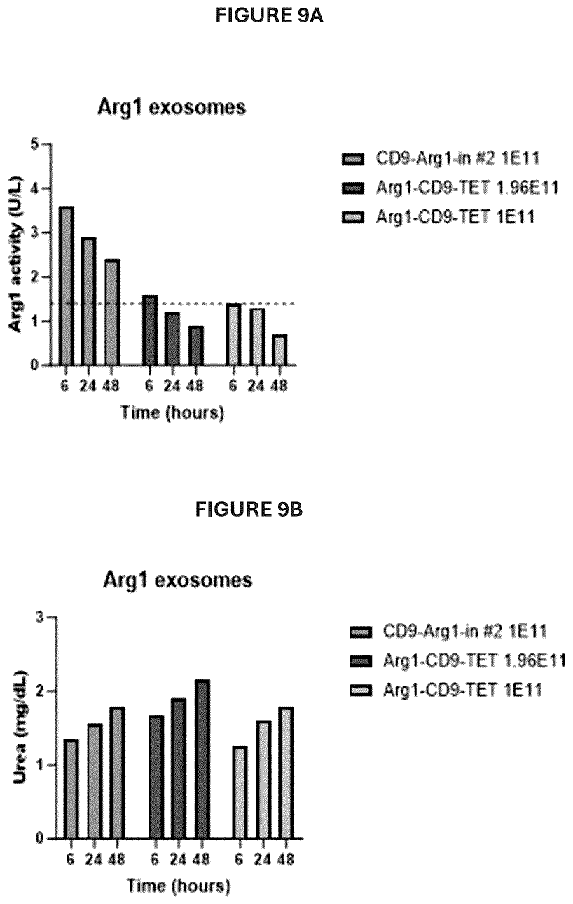

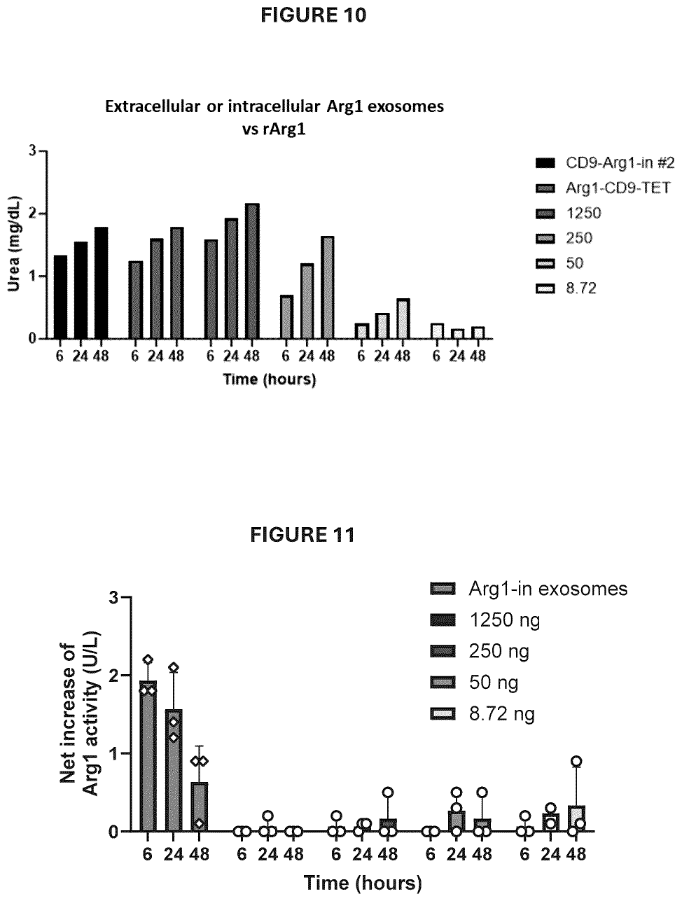

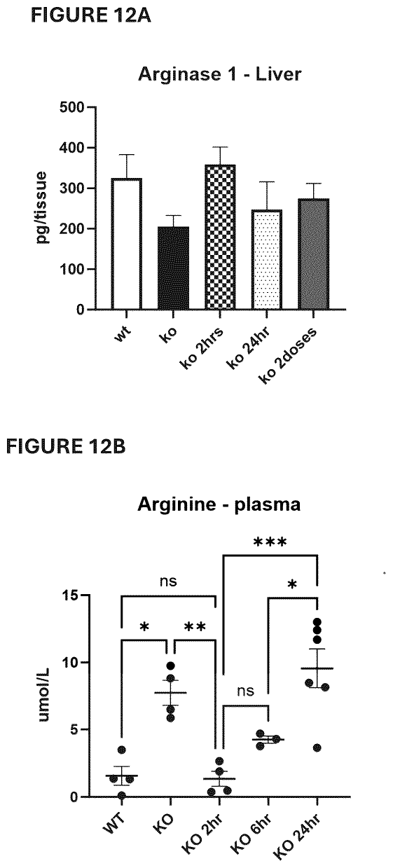

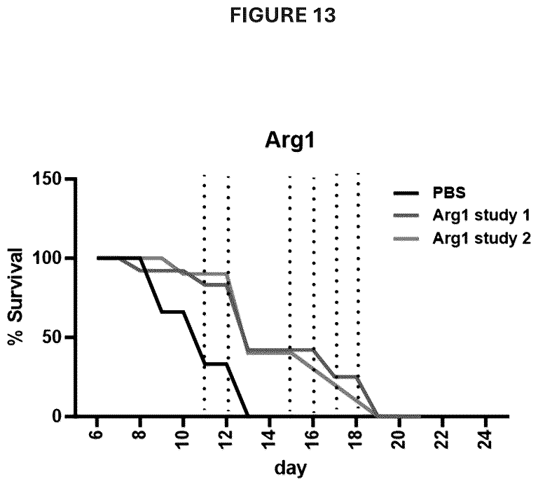

For example, exosomes (and other extracellular vesicles) may be produced via 293F cells. The 293F cells may be transfected with (or transduced with a lentivirus bearing) a polynucleotide that encodes a spike protein or a nucleocapsid protein, or chimeral fusions thereof, as described herein (see FIG. 3A), and expressing the spike protein or nucleocapsid protein, such that the spike protein or nucleocapsid is sorted into and displayed in or on the exosomes isolated therefrom. An example procedure for making exosomes from 293F cells may include steps as follows: 293F cells (Gibco™, Cat. #51-0029, ThermoFisher Scientific, Waltham, MA) may be tested for pathogens and found to be free of viral (cytomegalovirus, human immunodeficiency virus I and II, Epstein Barr virus, hepatitis B virus, and parvovirus B19) and bacterial (Mycoplasma) contaminants. Cells may be maintained in FreeStyle™ 293 Expression Medium (Gibco, Cat. #12338-018, ThermoFisher Scientific, Waltham, MA) and incubated at 37° C. in 8% CO2. For exosome production, 293F cells may be seeded at a density of 1.5E6 cells/ml in shaker flasks in a volume of about ¼ the flask volume and grown at a shaking speed of about 110 rpm. HEK293 cells may be grown in Dulbecco's modified Eagle's medium (DMEM) supplemented with 10% fetal calf serum.

To purify exosomes, the 293F cells may be cultured in shaker flasks for a period of three days. Cells and large cell debris may be removed by centrifugation at 300×g for 5 minutes followed by 3,000×g for 15 minutes. The resulting supernatant may be passed through a 0.22 μm sterile filtration filter unit (Thermo Fisher, Cat. #566-0020) to generate a clarified tissue culture supernatant (CTCS). The CTCS may be concentrated by centrifugal filtration (Centricon Plus-70, Ultracel-PL Membrane, 100 kDa size exclusion, Millipore Sigma, Cat. #UFC710008, St. Louis, MO), with about120 mL CTCS concentrated to about 0.5 mL. Concentrated CTCS my then be purified by size exclusion chromatography (SEC) in 1×PBS (qEV original columns/35 nm: Izon Science, Cat. #SP5), with the exosomes present in each 0.5 ml starting sample eluting in three 0.5 ml fractions. Purified exosomes may be reconcentrated using Amicon® Ultra-4 100 kDa cutoff spin columns (Cat. #UFC810024). This process may yield a population of exosomes/small EVs that have the expected ultrastructure and size distribution profile of human exosomes and contain the exosomal marker proteins CD9 and CD63, at a concentrating effect of about 500-fold, to a final concentration of 1E10-2E12 exosomes/ml. The concentration and size of the isolated extracellular vesicles may be measured using NANOSIGHT nanoparticle tracking analysis system (Malvern Panalytical, Malvern, UK).

The term “nucleic acid” refers to polynucleotides such as deoxyribonucleic acid (DNA) or ribonucleic acid (RNA), as well as chemically modified nucleic acids such as morpholino (PMO), peptide nucleic acid (PNA), 2′O-methyl, 2′ methoxy-ethyl, phosphoramidate, methylphosphonate, and phosphorothioate. Nucleic acids may be of any size. Nucleic acids include but are not limited to genomic DNA, cDNA, mRNA, iRNA, miRNA, RNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid might be employed for introduction into, e.g., transfection of, cells, e.g., in the form of RNA which can be prepared by in vitro transcription from a DNA template. The RNA can moreover be modified before application by stabilizing sequences, capping, and polyadenylation. Generally, nucleic acid can be extracted, isolated, amplified, or analyzed by a variety of techniques such as those described by, e.g., Green and Sambrook, Molecular Cloning: A Laboratory Manual (Fourth Edition), Cold Spring Harbor Laboratory Press, Woodbury, NY 2,028 pages (2012).

The terms “peptide”, “polypeptide” and “protein” are used interchangeably herein and refer to any chain of at least two amino acids, linked by a covalent chemical bound. As used herein a peptide can refer to the complete amino acid sequence coding for an entire protein or to a portion thereof. A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

As used herein, the phrase “protein polypeptide” means a polypeptide sequence of or derived from a protein. For example, a CD9 protein polypeptide may be any polypeptide of the CD9 protein, such as, e.g., a full length CD9 protein, a transmembrane domain polypeptide of a CD9 protein, a C-terminal stretch of a CD9 protein, an extracellular loop region of a CD9 protein, the intracellular (intralumenal) loop region of a CD9 protein, a C-terminal stretch of a CD9 protein, combinations thereof, and/or the like. Here, a protein polypeptide may be at least 10 amino acids long.

As used herein, the term “tetraspanin,” “tetraspanin protein,” or “tetraspanin polypeptide” means a molecule, especially a protein or polypeptide, containing four transmembrane domains or is capable of passing through or across a membrane four times. Tetraspanins include transmembrane 4 superfamily (TM4SF) proteins, including, but not limited to TSPAN1 through -TSPAN33, TSP-1, TSP-2, TSP-3, TSP-4, TSP-5, TSP-6, CD231, CO-029, NET-5, OCULOSPANIN, CD151-like, NET-2, NET-6, TSPAN14, NET-7, TM4-B, TSPAN17, TSPAN18, TSPAN19, UP1b (UPK1B), UP1a (UPK1A), RDS (PRPH2), ROM1, CD151, CD53, CD37, CD82, CD81, CD9, CD63, SAS, TSSC6, and TSPAN33. For a review of tetraspanins, see Susa et al., “Tetraspanins: structure, dynamics, and principles of partner-protein recognition,” Trends in Cell Biology, Volume 34, Issue 6, June 2024, Pages 509-522.

In a preferred embodiment, a useful tetraspanin is CD9. Here, CD9 includes but is not limited to any CD9, any eukaryote CD9, any animal CD9, any metazoan CD9, any chordate CD9, any vertebrate CD9, any mammalian CD9, any primate CD9, any hominid CD9, any human CD9, any CD9 having at least 90% identity at the amino acid level to human CD9 across at least 95% of the amino acid sequence, any CD9 having at least 90% identity at the amino acid level to human CD9 having an amino acid sequence set forth in GenBank accession number NP_001400172.1 across at least 95% of the amino acid sequence, any CD9 having at least 90% identity at the amino acid level to human CD9 having an amino acid sequence set forth in GenBank accession number NP_001400172.1 across at least 95% of the amino acid sequence as determined by basic local alignment search tool (BLAST®) or EXPASY SIM alignment tool for protein sequences, or any chimera thereof.

As used herein, the term “arginase activity” or “arginase” refers to enzymatic activity catalyzing the hydrolysis of arginine into urea and ornithine leading to the metabolism of ammonia and production of urea. As used herein, “arginase activity” also refers to the protein (enzyme) arginase, including Arg1. Here, arginase activity may be measured inter alia by urea production or ammonia reduction, and/or protein detection such as by antibody or other physicochemical methods. Arginase or arginase activity includes but is not limited to any arginase, any eukaryote arginase, any animal arginase, any metazoan arginase, any chordate arginase, any vertebrate arginase, any mammalian arginase, any primate arginase, any hominid arginase, any human arginase, any human arginase-1, any arginase having at least 90% identity at the amino acid level to human arginase-1 across at least 95% of the amino acid sequence, any arginase having at least 90% identity at the amino acid level to human arginase-1 having an amino acid sequence set forth in GenBank accession number CAA31188.1 across at least 95% of the amino acid sequence, any arginase having at least 90% identity at the amino acid level to human arginase-1 having an amino acid sequence set forth in GenBank accession number CAA31188.1 across at least 95% of the amino acid sequence as determined by basic local alignment search tool (BLAST®) or EXPASY SIM alignment tool for protein sequences, or any chimera thereof.

The term “transmembrane domain” or “TM” or “TM domain” means a portion of a polypeptide or a peptide containing hydrophobic amino acids, beta sheets, and/or alpha helices. A transmembrane domain is capable of being positioned within a membrane lipid bilayer. Transmembrane domains may contain about 20 to 25, usually 24 amino acids. Here, a useful transmembrane domain may be synthetic or natural, such as for example a transmembrane domain of a tetraspanin or other membrane protein. In one embodiment, a useful transmembrane domain is a CD8 transmembrane domain, such as for example a transmembrane domain having a primary structure as set forth at SEQ ID NO:7. Useful transmembrane domains can be predicted using various algorithms, such as but not limited to a TMHMM algorithm based on a hidden Markov model (Krogh A, Larsson B, von Heijne G, Sonnhammer EL. Predicting transmembrane protein topology with a hidden Markov model: application to complete genomes. J Mol Biol. 2001 Jan. 19; 305 (3): 567-80. doi: 10.1006/jmbi.2000.4315. PMID: 11152613). For a review of transmembrane domains, see Gunaa and Hegde, “Transmembrane Domain Recognition during Membrane Protein Biogenesis and Quality Control,” Control Current Biology 28, R498-R511, Apr. 23, 2018, and von Heijne, G., “Membrane-protein topology,” Nat Rev Mol Cell Biol 7, 909-918 (2006).

As used herein, “chimera,” “chimeric,” “chimeric protein,” and “chimeral protein” mean a synthetic polypeptide or protein containing parts of two or more proteins, peptides, polypeptides. Chimeral proteins can be produced by any methos, including but not limited to chemical synthesis and recombinant technology. A useful example of a chimeral protein includes a tetraspanin-enzyme fusion protein, such as but not limited to a CD9-arginase fusion protein of, from aminoterminus to carboxyterminus, (i) Arg1-transmembrane domain (TM)-CD9 (e.g. construct 1B), (ii) CD9-TM-Arg1 (e.g. construct 1C), and (iii) CD9-linker-Arg1 (e.g. construct 1D, CD9-Arg-in). Examples of useful primary structures of the above constructs (i) through (iii) include but are not limited to the sequences set forth in SEQ ID NO: 1, SEQ ID NO:2, and SEQ ID NO:3, respectively, or sequences having at least 90% identity thereto over at least 95% of the sequence.

### Several Embodiments

Embodiment 1 is a composition comprising an extracellular vesicle and an arginase.

Embodiment 2 is a composition of embodiment 1, wherein the arginase is Arg1.

Embodiment 3 is a composition of embodiment 1 or embodiment 2, wherein the extracellular vesicle is loaded with the arginase.

Embodiment 4 is a composition of any one of embodiments 1-3, wherein the arginase is positioned in the lumen of the extracellular vesicle.

Embodiment 5 is a composition of any one of embodiments 1-4, wherein the arginase is not a chimeral protein.

Embodiment 6 is a composition of any one of embodiments 1-4, wherein the arginase is fused to a tetraspanin.

Embodiment 7 is a composition of any one of embodiments 1˜4 and 6, wherein the arginase is fused to the C-terminus of a tetraspanin or to a linker fused to the C-terminus of a tetraspanin.

Embodiment 8 is a composition of any one of embodiments 1-3, wherein the arginase is positioned outside the extracellular vesicle.

Embodiment 9 is a composition of any one of embodiments 1-3 and 8, wherein the arginase is fused to a transmembrane domain which is fused to a tetraspanin.

Embodiment 10 is a composition of any one of embodiments 1-3, 8, and 9, wherein the N-terminus of the arginase is fused to a transmembrane domain which is fused to the C-terminus of a tetraspanin.

Embodiment 11 is a composition of any one of embodiments 1-3, 8, and 9, wherein the C-terminus of the arginase is fused to a transmembrane domain which is fused to the N-terminus of a tetraspanin.

Embodiment 12 is a composition of any one of embodiments 6-11, wherein the tetraspanin is a CD9 or fragment thereof.

Embodiment 13 is a composition of any one of embodiments 1˜4 and 6-7, wherein the arginase-fused-to-the-tetraspanin chimera has an amino acid sequence of SEQ ID NO:3.

Embodiment 14 is a composition of any one of embodiments 1-3 and 8-10, wherein the arginase-fused-to-the-tetraspanin chimera has an amino acid sequence of SEQ ID NO:2.

Embodiment 15 is a composition of any one of embodiments 1-3 and 8, 9, and 11, wherein the arginase-fused-to-the-tetraspanin chimera has an amino acid sequence of SEQ ID NO: 1.

Embodiment 16 is a composition of any one of embodiments 1-15, wherein the composition comprises a plurality of extracellular vesicle containing an arginase.

Embodiment 17 is a composition of any one of embodiments 1-16, wherein the composition is a pharmaceutical composition for use in treating hyperammonemia in a subject in need thereof.

Embodiment 18 is a composition of any one of embodiments 1-17, wherein the composition is a pharmaceutical composition for use in treating arginase deficiency in a subject in need thereof.

Embodiment 19 is a composition of any one of embodiments 1-18 further comprising a pharmaceutically acceptable excipient.

Embodiment 20 is a composition of any one of embodiments 1-19 in a pharmaceutical dosage form containing about 1E9-1E15 extracellular vesicles, about 1E9, about 1E10, about 1E11, about 1E12, about 1E13, about 1E14, about 1E15, or any amount between 1E9 and 1E15 extracellular vesicles.

Embodiment 21 is a composition of any one of embodiments 1-20 in a pharmaceutical dosage form containing about 1E12 extracellular vesicles.

Embodiment 22 is a composition of any one of embodiments 1-21 in a pharmaceutical dosage form containing about 1 ng to about 1 mg arginase.

Embodiment 23 is a composition of any one of embodiments 1-22 in a pharmaceutical dosage form containing about 5 ng to about 500 ng arginase.

Embodiment 24 is a composition of any one of embodiments 1-23 in a pharmaceutical dosage form containing about 5 ng to about 300 ng arginase.

Embodiment 25 is a composition of any one of embodiments 1-24 in a pharmaceutical dosage form containing about 5 ng, about 10 ng, about 15 ng, about 20 ng, about 25 ng, about 30 ng, about 40 ng, about 50 ng, about 75 ng, about 100 ng, about 150 ng, about 200 ng, about 250 ng, about 300 ng arginase, or any amount within the range of 5 ng to 300 ng arginase, inclusively.

Embodiment 26 is a composition of any one of embodiments 1-25, wherein the extracellular vesicle is an exosome or wherein the extracellular vesicles are exosomes.

Embodiment 27 is a method of treating arginase 1 deficiency in a subject in need thereof comprising contacting a hepatocyte with a composition of any one of embodiments 1-25.

Embodiment 28 is a method of treating hyperammonemia in a subject in need thereof comprising contacting a hepatocyte with a composition of any one of embodiments 1-25.

Embodiment 29 is a method of embodiment 27 or embodiment 28, wherein the hepatocyte is in the subject.

Embodiment 30 is a method of any one of embodiments 27-29 comprising administering the composition to a subject in need by intravenous administration.

Embodiment 31 is a method of making a composition of any one of embodiments 1-25 comprising transfecting a cell or cells with a polynucleotide construct encoding an arginase or arginase-tetraspanin chimera, culturing the transfected cell or cells in media, and harvesting from the media exosomes secreted from the cells.

Embodiment 32 is a method of embodiment 31, wherein the cell or cells is/are 293F cells.

What is claimed is:

1. A composition comprising an exosome and an arginase.

2. The composition of claim 1, wherein the arginase is Arg1.

3. The composition of claim 1, wherein the exosome is loaded with the arginase.

4. The composition of claim 1, wherein the arginase is positioned in the lumen of the exosome.

5. The composition of claim 1, wherein the arginase is not a chimeral protein.

6. The composition of claim 1, wherein the arginase is fused to a tetraspanin.

7. The composition of claim 6, wherein the arginase is fused to the C-terminus of a tetraspanin or to a linker fused to the C-terminus of a tetraspanin to form a first chimera.

8. The composition of claim 1, wherein the arginase is positioned outside the exosome.

9. The composition of claim 8, wherein the arginase is fused to a transmembrane domain which is fused to a tetraspanin.

10. The composition of claim 9, wherein the N-terminus of the arginase is fused to a transmembrane domain which is fused to the C-terminus of a tetraspanin to form a second chimera.

11. The composition of claim 9, wherein the C-terminus of the arginase is fused to a transmembrane domain which is fused to the N-terminus of a tetraspanin to form a third chimera.

12. The composition of claim 6, wherein the tetraspanin is a CD9 or fragment thereof.

13. The composition of claim 7, wherein the first chimera has an amino acid sequence of SEQ ID NO:3.

14. The composition of claim 10, wherein the second chimera has an amino acid sequence of SEQ ID NO:2.

15. The composition of claim 11, wherein the third chimera has an amino acid sequence of SEQ ID NO:1.

16. A pharmaceutical composition comprising:
a. a plurality of exosomes loaded with arginase-tetraspanin chimera; and
b. a pharmaceutically acceptable excipient.

17. The composition of claim 16 comprising about 1E9-1E15 exosomes, about 1E9, about 1E10, about 1E11, about 1E12, about 1E13, about 1E14, about 1E15, or any amount between 1E9 and 1E15 exosomes.

18. The composition of claim 16 in a pharmaceutical dosage form containing about 1 ng to about 1 mg arginase, about 5 ng to about 500 ng arginase, about 5 ng to about 300 ng arginase, about 5 ng, about 10 ng, about 15 ng, about 20 ng, about 25 ng, about 30 ng, about 40 ng, about 50 ng, about 75 ng, about 100 ng, about 150 ng, about 200 ng, about 250 ng, about 300 ng arginase, or any amount within the range of 5 ng to 300 ng arginase, inclusively.

19. A method of treating arginase 1 deficiency or hyperammonemia in a subject in need thereof comprising contacting a hepatocyte with a composition of claim 16.

20. The method of claim 19 comprising administering the composition to the subject in need by intravenous administration.


SEQUENCE LISTING (7 sequences)

SEQ ID NO: 1 - protein, 638 aa, synthetic construct
SAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPKKPTTTPAPRPPTPAPTIASQPLSLRPEACRPAAGGAVHTRGLDFACDIYIWAPLAGTCGVLLLSLVITLYCNHRNGGGGSGGGGSGGGGSPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 2 - protein, 639 aa, synthetic construct
MPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMVGGGGSGGGGSGGGGSIYIWAPLAGTCGVLLLSLVITLYCNHRNKPTTTPAPRPPTPAPTIASQPLSLRPEACRPAAGGAVHTRGLDFACDSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 3 - protein, 564 aa, synthetic construct
MPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMVGGGGSGGGGSGGGGSSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 4 - DNA, 1914 bp, synthetic construct
  source 1..1914: mol_type = other DNA
agtgctaagagtagaacgattggtatcattggggcgccttttagtaaaggtcagcctagaggaggagttgaggaaggacccactgtgctgcggaaggcggggctgttggagaagttgaaagagcaagagtgcgacgttaaggactatggcgacttgccattcgcagatataccgaacgattccccgtttcagatcgtgaagaatccgaggtctgttgggaaagcgtctgaacagctcgccggcaaagtggcggaggtaaaaaagaatggacgcattagcctggtgctcggtggagaccactctttggctatcgggtccatcagtgggcacgcacgggttcacccagaccttggtgtaatctgggttgatgctcatacggatattaatacaccattgaccacaacatccgggaatctgcatggtcaaccagtaagtttccttcttaaggaactcaaaggaaaaataccggatgtaccaggtttttcttgggtgacaccgtgcatctcagctaaagacatagtgtacatcggcctgcgggacgtagatcccggtgagcattacatcctcaaaactttgggcattaaatacttttctatgacggaagttgacagacttggcatcggcaaagttatggaagaaacacttagttacttgctggggcgaaagaaaagaccaattcacctctccttcgatgttgatgggctcgacccaagttttacgccagcgactgggactcccgtggtaggaggccttacctaccgcgagggtttgtacattacagaggaaatatacaagaccggtcttctttcaggactcgatatcatggaagtcaacccttctctcggaaagaccccagaagaggtgacacgcactgttaacactgcggtggcgattactttggcctgcttcgggttggctcgggaaggtaaccacaagccaatagattatttgaatccgccaaaaaagcccaccaccacccctgcccctagacctccaaccccagcccctacaatcgccagccagcccctgagcctgaggcccgaagcctgtagacctgccgctggcggagccgtgcacaccagaggcctggatttcgcctgcgacatctacatctgggcccctctggccggcacctgtggcgtgctgctgctgagcctggtcatcaccctgtactgcaaccaccggaatggcggaggcggaagcggaggcggaggctccggcggaggcggaagcccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtc

SEQ ID NO: 5 - DNA, 1917 bp, synthetic construct
  source 1..1917: mol_type = other DNA
atgccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtcggcggaggcggaagcggaggcggaggctccggcggaggcggaagcatctacatctgggcccctctggccggcacctgtggcgtgctgctgctgagcctggtcatcaccctgtactgcaaccaccggaataagcccaccaccacccctgcccctagacctccaaccccagcccctacaatcgccagccagcccctgagcctgaggcccgaagcctgtagacctgccgctggcggagccgtgcacaccagaggcctggatttcgcctgcgacagtgctaagagtagaacgattggtatcattggggcgccttttagtaaaggtcagcctagaggaggagttgaggaaggacccactgtgctgcggaaggcggggctgttggagaagttgaaagagcaagagtgcgacgttaaggactatggcgacttgccattcgcagatataccgaacgattccccgtttcagatcgtgaagaatccgaggtctgttgggaaagcgtctgaacagctcgccggcaaagtggcggaggtaaaaaagaatggacgcattagcctggtgctcggtggagaccactctttggctatcgggtccatcagtgggcacgcacgggttcacccagaccttggtgtaatctgggttgatgctcatacggatattaatacaccattgaccacaacatccgggaatctgcatggtcaaccagtaagtttccttcttaaggaactcaaaggaaaaataccggatgtaccaggtttttcttgggtgacaccgtgcatctcagctaaagacatagtgtacatcggcctgcgggacgtagatcccggtgagcattacatcctcaaaactttgggcattaaatacttttctatgacggaagttgacagacttggcatcggcaaagttatggaagaaacacttagttacttgctggggcgaaagaaaagaccaattcacctctccttcgatgttgatgggctcgacccaagttttacgccagcgactgggactcccgtggtaggaggccttacctaccgcgagggtttgtacattacagaggaaatatacaagaccggtcttctttcaggactcgatatcatggaagtcaacccttctctcggaaagaccccagaagaggtgacacgcactgttaacactgcggtggcgattactttggcctgcttcgggttggctcgggaaggtaaccacaagccaatagattatttgaatccgccaaaa

SEQ ID NO: 6 - DNA, 1692 bp, synthetic construct
  source 1..1692: mol_type = other DNA
atgccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtcggcggaggcggaagcggaggcggaggctccggcggaggcggaagcagtgctaagagtagaacgattggtatcattggggcgccttttagtaaaggtcagcctagaggaggagttgaggaaggacccactgtgctgcggaaggcggggctgttggagaagttgaaagagcaagagtgcgacgttaaggactatggcgacttgccattcgcagatataccgaacgattccccgtttcagatcgtgaagaatccgaggtctgttgggaaagcgtctgaacagctcgccggcaaagtggcggaggtaaaaaagaatggacgcattagcctggtgctcggtggagaccactctttggctatcgggtccatcagtgggcacgcacgggttcacccagaccttggtgtaatctgggttgatgctcatacggatattaatacaccattgaccacaacatccgggaatctgcatggtcaaccagtaagtttccttcttaaggaactcaaaggaaaaataccggatgtaccaggtttttcttgggtgacaccgtgcatctcagctaaagacatagtgtacatcggcctgcgggacgtagatcccggtgagcattacatcctcaaaactttgggcattaaatacttttctatgacggaagttgacagacttggcatcggcaaagttatggaagaaacacttagttacttgctggggcgaaagaaaagaccaattcacctctccttcgatgttgatgggctcgacccaagttttacgccagcgactgggactcccgtggtaggaggccttacctaccgcgagggtttgtacattacagaggaaatatacaagaccggtcttctttcaggactcgatatcatggaagtcaacccttctctcggaaagaccccagaagaggtgacacgcactgttaacactgcggtggcgattactttggcctgcttcgggttggctcgggaaggtaaccacaagccaatagattatttgaatccgccaaaa

SEQ ID NO: 7 - protein, 28 aa, Homo sapiens
IYIWAPLAGTCGVLLLSLVITLYCNHRN